(19) United States
(12) Patent Application Publication
(10) Pub. No.: US 2024/0345078 A1
(43) Pub. Date: Oct. 17, 2024

(54) ULTRASENSITIVE RAPID ANTIGEN DETECTION KIT AND DETECTION METHOD THEREOF

(71) Applicants: The University of Hong Kong, Hong Kong (HK); Advanced Biomedical Instrumentation Centre Limited, Hong Kong (HK)

(72) Inventors: Yang CAO, Hong Kong (HK); Ho Cheung SHUM, Hong Kong (HK); Haisong LIN, Hong Kong (HK)

(21) Appl. No.: 18/633,513

(22) Filed: Apr. 12, 2024

Related U.S. Application Data
(60) Provisional application No. 63/496,024, filed on Apr. 14, 2023.

(51) Int. Cl. G01N 33/543; G01N 33/569

(52) U.S. Cl. CPC G01N 33/54388; G01N 33/56983; G01N 2333/11; G01N 2333/135; G01N 2333/165

(57) ABSTRACT

The present invention relates to an ultrasensitive rapid antigen detection kit for detecting N protein from SARS-CoV-2 virus in a sample, and methods for detecting N protein. The kit includes a sample collector, a collection and enrichment tube and a rapid antigen detection component with a test strip. The collection and enrichment tube contains a first part containing lyophilized ATPS reagents and a second part containing a lysis buffer. The sample is mixed with the lyophilized ATPS reagents and the lysis buffer to form a mixture. The enrichment is realized by concentrating the target analyte into one of the two phases formed by ATPS.

### FIELD OF THE INVENTION

The present invention generally relates to the field of virus detection. More specifically, the present invention provides a phase separation-assisted preconcentration method for ultrasensitive rapid antigen tests.

### BACKGROUND OF THE INVENTION

The outbreak of COVID-19 has accelerated the development and deployment of the SARS-CoV-2 LFTs. The LFA-based rapid antigen test (LFA-RAT) has played an essential role in monitoring the spread of disease and cutting off the transmission routes. Lateral flow assay (LFA) enables fast identification of analytes with good user-friendliness and low implementation expense. Hence, it is more suitable for large-scale screening than quantitative polymerase chain reaction (qPCR) due to its shorter sample-to-result time, more straightforward processing steps, and higher screening capacity.

Although the post-pandemic era has arrived, investigations and contributions to the coronavirus research and diagnosis studies are still carrying on. For pandemics like COVID-19, whose infectious viruses transmit at an exponential rate at the early stage, the most effective detection strategies should be accurate tests that are simple, low-cost, and rapid. Though the RT-PCR test is highly sensitive to monitoring the virus load, its detection efficiency is limited by several inherent drawbacks including complexity, high cost, and long process time.

The lateral flow assay-based rapid antigen test (LFA-RAT) has the potential to meet the demands for efficient and frequent screening of virus transmission. However, their reliability is limited due to poor performance when detecting samples with ultra-low concentrations. Current LFA-RAT cannot distinguish the viral loads with Ct value larger than 30, which relates to the early-stage spread of diseases. The limited accuracy of LFA-RAT hinders its reliability among asymptomatic carriers with ultralow viral loads. The low sensitivity issues of LFA-RAT, mainly caused by the low analyte concentrations, are still worth solving to make this technique well-prepared for possible future pandemics and other infectious diseases. To be more ambitious, if the sensitivity and limit of detection (LOD) of LFA-RAT are comparable to the standard PCR, the LFA-RAT can be a perfect point-of-care diagnostic technique to replace PCR and be widely used in resources-limited settings.

When considering the enzymatic reaction in confined and dynamic compartments formed by phase separation, different types of phase separation have been investigated. While some researchers have tried the aqueous two-phase system (ATPS) to modify the lateral flow assays previously¹⁻³, the enzymatic reactions in aqueous two-phase systems (ATPSs), a representative segregative-type phase separation, are less investigated. Especially, when reaction-related biomolecules have various partitioning affinities in ATPSs, whether the ATPSs will enhance or hinder the enzymatic reaction, has not been studied clearly. Moreover, current studies are focused on equilibrium ATPSs,²⁵ research addressing the complex interactions between enzymatic reactions and non-equilibrium ATPSs remains relatively limited.

There exists a necessity within the field to improve the existing lateral flow assay-based rapid antigen test. The present invention addresses this need.

### SUMMARY OF THE INVENTION

Antigen sample preconcentration is a promising approach to improve the sensitivity and limit of detection (LOD) of rapid antigen tests. The present invention uses a non-equilibrium enzyme-loaded aqueous two-phase system (ATPS) via phase separation-assisted preconcentration (PSAP) technology to enrich the sample concentration from an undetectable range to a detectable one, thus improving the sensitivity and limit of detection (LOD) of lateral flow assay-based rapid antigen test (LFA-RAT) and making its sensitivity comparable with the PCR tests. The enrichment is realized by concentrating the target analyte into one of the two phases formed by ATPS. FIGS. 1A-1B depicts a schematic diagram of the ATPS-enhanced ultrasensitive rapid antigen detection process. The sample collection and enrichment processes are integrated into a point-of-care device that can be directly used by the existing LFA-RAT products.

In a first aspect, the present invention provides an ultrasensitive rapid antigen detection kit for detecting N protein from RNA viruses in a sample. The kit includes a sample collector for collecting the sample from a patient, a collection and enrichment tube, a rapid antigen detection component comprising a test strip, and a manual. The collection and enrichment tube includes a first part containing a lyophilized reagent, and a second part containing a lysis buffer. The sample is mixed with the lyophilized reagent and the lysis buffer to form at least one Janus droplet having enriched N protein.

The test strip comprises a sample application port for applying the at least one Janus droplet from the collection and enrichment tube to the test strip, an absorbent pad for wicking the sample, a detection line for capturing the N protein, and a control line for indicating the validity of the detection.

In one embodiment, the lyophilized reagent includes at least two polymers or a polymer containing a salt (Polymer: PEG, Ficoll, dextran; Salt: sodium carbonate, sodium citrate, potassium phosphate etc.). A first polymer of the at least two polymers is polyethylene glycol with a molecule weight ranging from 1000 Da to 40,000 Da; and a second polymer of the at least two polymers is dextran with a molecule weight ranging from 8,000 Da to 100,000 Da, Ficoll with a molecule weight ranging from 50,000 Da to 400,000 Da, or a salt sodium citrate with a molecule weight of ranging from 100 Da to 400 Da, or a combination thereof.

The first polymer forms a hydrophobic upper phase, the second polymer forms a hydrophilic bottom phase, and the N protein is attracted by the hydrophilic bottom phase. The hydrophobic upper phase and the hydrophilic bottom phase are combined by a droplet microfluidic device with two flow-focusing junctions to generate the at least one Janus droplet. The mass fraction of the PEG phase is in a range of 2-4% (w/w). The mass fraction of the dextran phase is in a range of 16-20% (w/w).

In another embodiment, the hydrophobic upper phase further contains 2 mM to 10 mM of 10-Acetyl-3,7-dihydroxyphenoxazine and H₂02, and the hydrophilic bottom phase further comprises 5 pM to 5000 pM of HRP-conjugated antibody.

In one embodiment, the first polymer and the second polymer have a volume ratio in a range of 1:1 to 29:1. The kit enhances an enrichment ratio of the N protein with a range of 2 to 5.

In one embodiment, the at least one Janus droplet has a diameter of 50-70 m, resulting in a total droplet volume of 150 pL.

In one embodiment, the lysis buffer includes Triton X, sodium dodecyl sulfate (SDS) lysis buffer with dithiothreitol (DTT).

In one embodiment, the RNA viruses include coronaviruses, influenza viruses, Monkeypox viruses, human immunodeficiency viruses, respiratory syncytial viruses and lymphocytic choriomeningitis viruses.

In one embodiment, the kit is capable to discern samples with a Ct value of approximately 40.3.

In a second aspect, the present invention provides a method for detecting N protein from RNA viruses in a sample. The method includes collecting a sample from a subject suspected of being infected with the RNA viruses; mixing the sample with a lyophilized reagent and a lysis buffer (with the ratio within the range of 1:1 to 1:5) in a collection and enrichment tube to form at least one Janus droplet having enriched N protein; and applying the at least one Janus droplet from the collection and enrichment tube to a test strip of a rapid antigen detection component to obtain the test result.

In one embodiment, the lyophilized reagent includes at least two polymers or a polymer containing a salt (Polymer: PEG, Ficoll, dextran; Salt: sodium carbonate, sodium citrate, potassium phosphate etc.). A first polymer of the at least two polymers is polyethylene glycol with a molecule weight ranging from 1000 Da to 40,000 Da; and a second polymer of the at least two polymers is dextran with a molecule weight ranging from 8,000 Da to 100,000 Da, Ficoll with a molecule weight ranging from 50,000 Da to 400,000 Da, or a salt sodium citrate with a molecule weight of ranging from 100 Da to 400 Da, or a combination thereof.

The first polymer forms a hydrophobic upper phase, the second polymer forms a hydrophilic bottom phase, and the N protein is attracted by the hydrophilic bottom phase, and wherein the hydrophobic upper phase and the hydrophilic bottom phase are combined by a droplet microfluidic device with two flow-focusing junctions to generate single-phase droplet.

In one embodiment, the first polymer and the second polymer have a volume ratio in a range of 1:1 to 29:1.

In one embodiment, the kit enhances an enrichment ratio of the N protein with a range of 2 to 5.

In one embodiment, the lysis buffer includes Triton X, sodium dodecyl sulfate (SDS) lysis buffer with dithiothreitol (DTT).

In one embodiment, the RNA viruses include coronaviruses, influenza viruses, Monkeypox viruses, human immunodeficiency viruses, respiratory syncytial viruses and lymphocytic choriomeningitis viruses.

The present invention systematically investigates the compositional factors of the PSAP technology including types of phase separation polymers, buffer pH, polymer molecular weights, mass ratios, and volume ratios, to explore their effect on the antigen enrichment performance and ensure assay fluidity when interacting with RAT strips.

The results confirm that the catalysis first happens at the aqueous interfaces between polyethylene glycol (PEG) and dextran due to the different partitioning affinities of enzymes and substrates. Besides, the results also indicate that PEG can diminish reaction rates under both high and low enzyme loads. However, when enzyme loading is high, reaction activity can be sustained. Further investigation delves into the microscale interfacial phenomena within enzyme-loaded ATPS droplets. By adjusting the initial compositions of these active droplets, a variety of phase separation patterns are observed. These discoveries contribute to the advancement of understanding non-equilibrium ATPSs and their involvement in enzymatic reactions, thereby paving the way for new research directions and applications in biotechnology and associated domains.

The present invention has the following advantages: (1) The proposed ATPS compositions can be directly applied to commercial LFA products, without additional modification on the existed test strips; and (2) The new design of sample handling bottle can collect the sample, lysis the virus and rehydrate the lyophilized ATPS reagents simultaneously.

### BRIEF DESCRIPTION OF THE DRAWINGS

Embodiments of the invention are described in more details hereinafter with reference to the drawings, in which:

FIG. 1A shows the collection of antigens from saliva or a swab, followed by ATPS-based treatment to enrich the antigens. FIG. 1B shows a sensitivity comparison between the initial (C₁) and enriched (C₂) antigen concentrations;

FIG. 2 depicts a schematic diagram showing the phase separation of antigen and ATPS reagents, and the definition of distribution ratio (DR). Standard error bars represent triplicate experiments;

FIG. 3 depicts calibration curves of resorufin dissolving in various buffer environments;

FIG. 4A shows a schematic illustrating the generation of ATPS droplets using a microfluidic device. FIG. 4B shows a schematic depicting the single-phase (below the binodal curve) and two-phase (above binodal curve) statues of an enzyme-loaded ATPS droplet;

FIG. 5A shows a reaction scheme illustrating HRP catalyzing the conversion of ADHP into resorufin. FIG. 5B depicts partitioning preferences of HRP-dAb and HRP in PEG-dextran ATPS. FIG. 5C depicts partitioning preferences of ADHP and resorufin in PEG-dextran ATPS. FIG. 5D shows a schematic showing the transition from bulk phase reaction (Left, before phase separation) to interfacial reaction (Right, after phase separation) in an enzyme-loaded non-equilibrium ATPS, and a zoom-in view illustrating the interfacial catalysis process of ADHP into resorufin;

FIG. 6 shows resorufin distribution in Janus PEG-dextran droplets. Scale bar: 100 μm;

FIGS. 7A-7F depict distribution ratios of SARS-CoV-2 nucleocapsid protein in ATPSs by adjusting different parameters, including buffer pH values, ATPS types, PEG molecular weights, dextran molecular weights, PEG mass fractions, and dextran mass fractions, respectively. Standard error bars represent triplicate experiments;

FIG. 8 summarizes the distribution ratios (DRs) when modifying different ATPS parameters. Standard error bars represent triplicate experiments;

FIGS. 9A-9C depict linear temporal trends of resorufin production at different dextran and PEG concentrations when enzyme load is 5.56 pM;

FIGS. 10A-10C depict Linear-and-plateau temporal trends in resorufin production at varying dextran and PEG concentrations when enzyme load is 555.56 pM;

FIG. 11A shows schematics showing the PEG-to-dextran volumetric ratios. FIG. 11B depicts the calculated enrichment ratios in ATPSs with different volumetric ratios. Standard error bars represent triplicate experiments;

FIG. 12 depicts standard calibration curve of N protein dilutions in dextran-rich solution. The error bars are calculated based on triplicate independent experiments. Standard error bars represent triplicate experiments;

FIG. 13A shows a schematic illustration showing the nucleation and growth of PEG-rich sub-droplets from the initially homogeneous mixture. FIG. 13B shows time series snapshots of a droplet undergoing simultaneous enzymatic reaction and phase separation (Scale bar: 35 μm, the dextran-rich phase is fluorescently labeled by FITC-dextran);

FIG. 14A shows a schematic representation depicting the spinodal decomposition in proximity to the concentrated dextran-rich phase. FIG. 14B shows time series snapshots of a droplet undergoing phase separation when solely ADHP and H₂O₂ are involved (Scale bar: 35 μm, the dextran-rich phase is fluorescently labeled by FITC-dextran);

FIG. 15A shows a schematic representation illustrating the spinodal decomposition in proximity to the concentrated dextran-rich phase. FIG. 15B shows time series snapshots of a droplet undergoing phase separation when solely HRP-dAb is involved (Scale bar: 35 μm, the dextran-rich phase is fluorescently labeled by FITC-dextran);

FIG. 16A shows a schematic depicting the common phase separation process of the ATPS mixture. FIG. 16B shows time series snapshots of a PEG-dextran droplet undergoing phase separation (Scale bar: 35 μm, the dextran-rich phase is fluorescently labeled by FITC-dextran);

FIG. 17 depicts assay flow performance when increasing the dextran mass fraction from 20% wt to 30% wt. Standard error bars represent triplicate experiments;

FIGS. 18A-18B show a schematic diagram illustrating the steps performed for single-phase control and ATPS enrichment experiments;

FIG. 19A shows the test line and control line images of commercial and ATPS-enhanced RAT strips. FIG. 19B shows signal intensities of the test lines with different SARS-CoV-2 N protein concentrations. Standard error bars represent triplicate experiments;

FIG. 20A shows the test line and control line images of commercial and ATPS-enhanced RAT strips. FIG. 20B depicts signal intensities of the test lines with different SARS-CoV-2 N protein concentrations. Standard error bars represent triplicate experiments;

FIG. 21A shows the test line and control line images of commercial and ATPS-enhanced RAT strips. FIG. 21B depicts signal intensities of the test lines with different SARS-CoV-2 N protein concentrations. Standard error bars represent triplicate experiments;

FIG. 22A shows the test line and control line images of commercial and ATPS-enhanced RAT strips. FIG. 22B depicts signal intensities of the test lines with different influenza A virus protein concentrations. Standard error bars represent triplicate experiments;

FIG. 23A shows the test line and control line images of commercial and ATPS-enhanced RAT strips. FIG. 23B depicts signal intensities of the test lines with different influenza B virus protein concentrations. Standard error bars represent triplicate experiments;

FIG. 24A shows a schematic illustrating the assay preparation in single-phase and ATPS groups. FIG. 24B depicts Ct values of samples with varying concentrations. Standard error bars represent triplicate experiments measured by RT-qPCR;

FIG. 25A shows the test line and control line images of ATPS and single-phase rapid antigen test strips (Genrui brand). FIG. 25B shows the test line signal intensities of different samples. Standard error bars represent triplicate experiments;

FIG. 26A shows the test line and control line images of ATPS and single-phase rapid antigen test strips. FIG. 26B depicts Ct values of the three samples; and

FIG. 27A shows a conceptual design of the ATPS-based enrichment module containing freeze-drying ATPS powder, and FIG. 27B shows the overall workflow thereof.

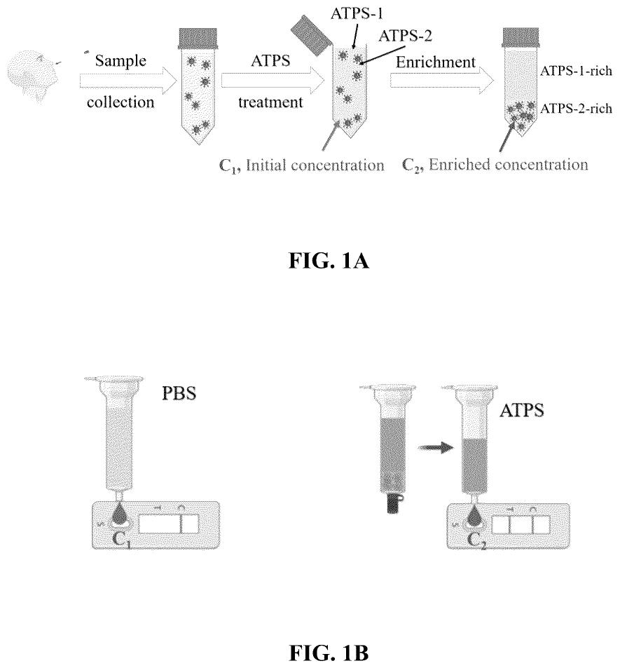

### DETAILED DESCRIPTION

Exploring the complex interplay between phase separation and enzymatic reactions poses significant challenges, particularly under non-equilibrium conditions. Systems that involve both phase separation and chemical reactions are characteristic examples of non-equilibrium systems. Understanding the interactions between phase separation and chemical reactions is crucial for advancing our knowledge of biological processes and exploring their potential applications in various fields.

The efficacy of rapid antigen tests in screening is hindered by low sensitivity and inadequate detection limits, primarily stemming from the low concentration of collected analytes.

In order to fill the huge gap, in a first aspect, the present invention provides an ultrasensitive rapid antigen detection kit for detecting a target antigen from viruses in a sample. The kit includes a sample collector for collecting the sample from a patient, a collection and enrichment tube, a rapid antigen detection component comprising a test strip, and a manual. By adjusting the two-phase volume ratio and property parameters of the ATPS (e.g., compositions, concentrations, molecular weights, etc), the undetectable analyte concentration can be enriched to a detectable range.

The collection and enrichment tube has two parts: one part contains lyophilized ATPS reagents, and the second part contains a lysis buffer. The test strip includes a sample application port for applying the mixture from the collection and enrichment tube to the test strip, an absorbent pad for wicking the sample, a detection line for capturing the antigen, and a control line for indicating the validity of the detection. The sample is mixed with the lyophilized ATPS reagents and the lysis buffer to form a mixture having enriched antigen.

Phase separation can facilitate the transportation of small molecules by providing aqueous interfaces. Compartmentalization after phase separation allows enzymes and substrates to be partitioned, increasing the system functionality and versatility and thereby modulating the reaction kinetics. The physically segregated but chemically interacting crowding environments construct a flexible platform for investigating the mass transfer and motility far from the equilibrium.

In one embodiment, the present invention provides an ultrasensitive rapid antigen detection kit for detecting N protein from RNA viruses in a sample.

In one embodiment, the present invention provides a non-equilibrium, enzyme-loaded aqueous two-phase system (ATPS) as a model system for elucidating the mechanisms of how phase separation influences the reaction activity, specifically in the context of horseradish peroxidase-conjugated protein (HRP-dAb) catalyzing the amplex red (10-Acetyl-3,7-dihydroxyphenoxazine, ADHP) into fluorescent resorufin. The reaction is occurred in aqueous environments constructed by polyethylene glycol (PEG) and dextran.

In one embodiment, the RNA viruses may include, but are not limited to, coronavirus, influenza viruses, Monkeypox viruses, human immunodeficiency viruses, respiratory syncytial viruses and lymphocytic choriomeningitis viruses.

Preferably, the viruses are SARS-CoV-2 viruses.

In another embodiment, the target antigen may include, but not limited to N protein.

In one embodiment, the lyophilized ATPS reagents comprise at least two polymers or a polymer containing a salt (Polymer: PEG, Ficoll, dextran; Salt: sodium carbonate, sodium citrate, potassium phosphate etc.). A first polymer of the at least two polymers is polyethylene glycol (PEG) with a molecule weight ranging from 1000 Da-40,000 Da; and a second polymer of the at least two polymers is dextran with a molecule weight ranging from 8,000 Da-500,000 Da, or Ficoll with a molecule weight ranging from 50,000 Da-400,000 Da, or a salt Sodium citrate with a molecule weight of ranging from 100 Da-400 Da, or a combination thereof. Not limited to the above, many polymers/salts can be used to form ATPS when their concentration is high enough.

In one embodiment, the present invention uses polyethylene glycol (PEG), dextran, and Ficoll to form the ATPS. In fact, many other polymers or salts can be used to form ATPS. In one embodiment, the molecule weight of PEG is in a range of 1000 Da-40,000 Da. Different molecule weights of PEG, dextran, Ficoll, and sodium citrate are tested and listed in the Table 1.

TABLE 1

| Polymer/salt | Molecule weight (Mw) |
|---|---|
| PEG | 35,000 Da; 20,000 Da; 10,000 Da; 8,000 Da; 6,000 |
| | Da; 4,000 Da; 3,500 Da; 3,350 Da; 2,000 Da |
| dextran | 500,000 Da; 70,000 Da; 40,000 Da; 10,000 Da |
| Ficoll | 400,000 Da; 70,000 Da |
| Sodium citrate | 294.1 Da |

In another embodiment, the present invention uses PEG and dextran or Ficoll to form the ATPS. Table 2 lists different concentrations of PEG (MW=20,000 Da) and dextran (MW=10,000 Da). Table 3 lists different concentrations of PEG (MW=35,000 Da) and Ficoll (MW=400,000 Da). Table 4 lists different concentrations of PEG (MW=20,000 Da) and sodium citrate (MW=294.1 Da).

TABLE 2

| | PEG 20,000 Da | dextran 10,000 Da |
|---|---|---|
| ATPS group No. | concentration(wt) | concentration(wt) |
| 1 | 2% | 18% |
| 2 | 2% | 20% |
| 3 | 3% | 18% |
| 4 | 3% | 20% |
| 5 | 4% | 18% |
| 6 | 4% | 20% |
| 7 | 5% | 18% |
| 8 | 5% | 20% |

TABLE 3

| | PEG 35,000 Da | Ficoll 400,000 Da |
|---|---|---|
| ATPS group No. | concentration(wt) | concentration(wt) |
| 9 | 7.5% | 10% |
| 10 | 3% | 20% |
| 11 | 4% | 20% |
| 12 | 5% | 20% |

TABLE 4

| | PEG 20,000 Da | Sodium citrate 294.1 Da |
|---|---|---|
| ATPS group No | concentration(wt) | concentration(wt) |
| 13 | 2% | 18% |
| 14 | 2% | 20% |
| 15 | 3% | 18% |
| 16 | 3% | 20% |
| 17 | 4% | 18% |
| 18 | 4% | 20% |

In one embodiment, the first polymer and the second polymer have a volume ratio in a range of 1:1 to 29:1. For instance, the first polymer and the second polymer have a volume ratio of 1:1, 4:1, 9:1, or 29:1.

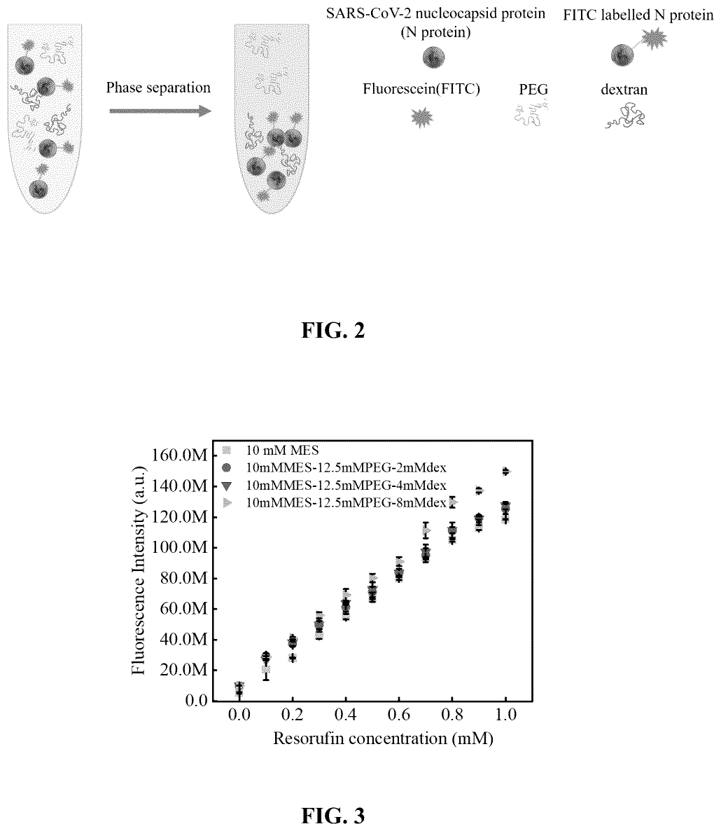

After phase separation, the first polymer forms a hydrophobic upper phase, the second polymer forms a hydrophilic bottom phase, and the N protein is attracted by the hydrophilic bottom phase, as shown in FIG. 2.

In a second aspect, the present invention also provides a method for detecting N protein from RNA viruses in a sample, including (a) collecting a sample from a subject suspected of being infected with the RNA viruses; (b) mixing the sample with lyophilized ATPS reagents and a lysis buffer in a collection and enrichment tube to form a mixture having enriched N protein; and (c) applying the mixture from the collection and enrichment tube to a test strip of a rapid antigen detection component to obtain the test result.

In particular, the present invention provides a method for detecting N protein from SARS-CoV-2 viruses in a sample, including (a) collecting a sample from a subject suspected of being infected with the SARS-CoV-2 viruses; (b) mixing the sample with lyophilized ATPS reagents and a lysis buffer in a collection and enrichment tube to form a mixture having enriched N protein; and (c) applying the mixture from the collection and enrichment tube to a test strip of a rapid antigen detection component to obtain the test result.

In summary, the present invention reveals that the macromolecular crowding effect of PEG and the substrate interaction mediated by dextran significantly influence the enzyme kinetics of HRP catalyzing Amplex red. When considering the enzyme-loaded ATPS droplets formed by the combination of PEG and dextran, the partitioning differences between the enzyme and substrate lead to adjustable reaction interfaces. This dynamic interplay ultimately alleviates the inhibitory effect that is observed when PEG and dextran are individually considered. These findings provide valuable insights into the complex interplay of macromolecular crowding, enzyme kinetics, and liquid-liquid phase separation in enzyme-loaded ATPS.

Besides, using the evaporating enzyme-loaded ATPS droplets, the non-equilibrium interfacial phenomena originated from the intertwined physics in enzymatic reaction and LLPS can be observed. The internal flow inside evaporating droplets can be enhanced by involving the reactants, and an accelerated LLPS is observed. Additionally, the order in which the sub-droplets appear during the phase separation process can be controlled by tuning the compositions of the enzyme-loaded ATPS droplet. The varying dynamic states suggest that the enzymatic reaction can control the thermodynamic pathways of phase separation. The coupling between phase separation and reaction could have played an important role in biological systems, such as liquid organelles, and inspires new ways to control reactions via LLPS, and vice versa.

### EXAMPLE

### Example 1—Materials and Methods

### Chemicals and Reagents

Polyethylene glycol (PEG) polymers with various molecule weights (Mw=8000 Da, 10000 Da, and Mw 20000 Da) and dextran (Mw=10000 Da, 70000 Da, 500000 Da), FITC-dextran (Mw 10,000 Da) and phosphate-buffered saline (50 mmol/L, pH=7.4) were purchased from Sigma-Aldrich; N-2-hydroxyethylpiperazine-N′-2-ethanesulfonic acid with various pH values (HEPES, pH=6.5, 7.4, 9.0) and phosphate-buffered saline (PBS, 50 mmol/L, pH=7.4) were purchased from Macklin. UltraPure™ distilled water (DNAse, RNAse, Free) was purchased from Invitrogen. The recombinant SARS-CoV-2 BA.2 nucleocapsid protein with his-tag was purchased from R & D Systems Hong Kong Limited. The FITC conjugation kit (Lightning-Link® (ab102884)) was purchased from Abcam. HFE 7500 oil was purchased from 3M, USA. The 008 fluor surfactant was purchased from RAN Biotechnologies, USA. HRP conjugated secondary antibody (HRP-dAb, Mw 180,000 Da, 1 mg/ml) was purchased from ThermoFisher. Amplex Red, 10-Acetyl-3,7-dihydroxyphenoxazine (Macklin, CAS No. 119171-73-2, ADHP, Mw 257.24 Da) was purchased from DIECKMANN(HK). Resorufin (Macklin, CAS No. 635-78-9, MW 213.19 Da) was purchased from DIECKMANN(HK). QuantaRed™ Enhanced Chemifluorescent HRP Substrate Kit was purchased from ThermoFisher. MES buffer (M885671, 0.2 M, pH 6.0) was purchased from DIECKMANN(HK).

### RATs Colorimetric Image Process and Analysis

iPhone (14 pro) was used to capture the RATs images under fixed light source conditions. Then, those color images were processed by Image J software to extract the gray values of “test” and “control” lines. Finally, sample concentrations could be reflected by the signal intensities. The LOD information could be obtained after plotting the test line signal intensities versus the antigen concentrations.

### Example 2

### Dilutions of Reconstituted SARS-CoV-2 N Protein

SARS-CoV-2 viruses have four structural proteins: spike protein (S protein), nucleotide protein (N protein), membrane protein (M protein), and envelope protein (E protein). These proteins have different sizes and loads in a single virus and possess specific genetic or replication functions. The S protein is the most abundant and is essential in recognizing and infecting the host cells. However, its rapid mutation makes the markets less willing to develop and fabricate diagnostics assays or kits targeting S protein, though targeting S protein can promote diagnostics specificity. In contrast, the second abundant N protein, with more stable structures among several virus variants, is the most used target antigen in commercialized rapid antigen test kits. Thus, the assays are developed based on the rapid antigen test kits that targeted the N protein testing.

N protein stock solutions were reconstituted using PBS (pH=7.4). Specifically, 100 μL of PBS (pH=7.4) was added to 100 μg of lyophilized protein powder to achieve a protein stock concentration of 1 μg/L. The reconstituted protein was stored in a 4° C. refrigerator and was tested within one month. In the subsequent experiments, N protein dilutions were prepared using various buffers and ATPS solutions, according to different parameters under investigation.

To measure the calibration curve of N protein, the reconstituted N protein was labelled using fluorescein isothiocyanate (FITC), following the standard protocol of protein (Lightning-Link®, ab102884, Abcam). Serial N protein dilutions were prepared using single-phase buffers and dextran-rich solutions separately. That is, the N protein solutions with various concentrations were prepared by serially diluting the stock N protein in the extracted dextran-rich phase. All the N proteins were relocated in 96-well plates and characterized by SpectraMax iD3 multi-mode microplate reader (Molecular Devices, CA).

The RATs for SARS-CoV-2 N protein dilutions were performed by pipetting a 100-L aliquoted N protein dilution to the sample loading area of the test strips. Both the single-phase and the ATPS-enriched sample solutions were prepared and tested within one day.

### Example 3

### Preparation of Three Types of ATPS

Many polymers/salts can form ATPS when their concentrations are sufficiently high. In this example, three sets of ATPSs were prepared as examples to demonstrate the general methodology when developing the ATPS-enhanced ultrasensitive rapid antigen tests: (1) Polyethylene glycol (PEG) and dextran, (2) PEG and Ficoll and (3) PEG and sodium citrate. In set (1), the concentration of PEG was in a range of 6.25 mM to 12.5 mM, and the concentration of dextran was in a range of 2 mM to 8 mM; in set (2), the concentration of PEG was in a range of 6.25 mM to 12.5 mM, and the concentration of Ficoll was in a range of 3 mM to 9 mM; in set (3), the concentration of PEG was in a range of 6.25 mM to 12.5 mM, and the concentration of sodium citrate was in a range of 5 mM to 14 mM.

The PEG-dextran mixture was prepared at a concentration of 10 wt % for each polymer by dissolving the polymer powders directly into two separate solvents: deionized water and a 10 mM MES buffer solution with a pH of 6.0. To achieve phase-separated ATPS, the mixtures containing PEG and dextran in their respective solvents were centrifuged at 9000 rpm for 45 minutes and subsequently allowed to stand undisturbed overnight. This process facilitated the formation of distinct PEG-rich and dextran-rich phases within each system.

The PEG-Ficoll mixture was prepared at a concentration of 8.5 wt % for each polymer by dissolving the polymer powders directly into two separate solvents: deionized water and a 10 mM MES buffer solution with a pH of 7.4. To achieve phase-separated ATPS, the mixtures containing PEG and Ficoll in their respective solvents were centrifuged at 9000 rpm for 45 minutes and subsequently allowed to stand undisturbed overnight. This process facilitated the formation of distinct PEG-rich and Ficoll-rich phases within each system.

The PEG-sodium citrate mixture was prepared at a concentration of 12.5 wt % for each polymer by dissolving the polymer powders directly into two separate solvents: deionized water and a 10 mM MES buffer solution with a pH of 8.0. To achieve phase-separated ATPS, the mixtures containing PEG and sodium citrate in their respective solvents were centrifuged at 9000 rpm for 45 minutes and subsequently allowed to stand undisturbed overnight. This process facilitated the formation of distinct PEG-rich and salt-rich phases within each system.

For each ATPS group, a 50 g stock solution was prepared by dissolving phase-forming materials in buffers. The resulting ATPS mixture was centrifuged for 70 minutes at 9000 rpm and statically settle down overnight. The phase-separated upper and bottom phases were carefully separated via pipette and recombined at series of upper-to-lower volumetric ratios in different experiments. All the working solutions were stored in 4° C. refrigerator and used up within one week.

### Example 4

### Design and Fabrication of Droplet Microfluidic Devices

To accurately control the composition of enzyme-loaded ATPS droplets and their corresponding control counterparts, a microfluidic chip featuring two flow-focusing junctions was designed, enabling the generation of water-water-oil ATPS Janus emulsions in a controllable manner. The microfluidic chip included three inlets and one outlet, and was fabricated by standard photolithography techniques.

The single-phase ATPS droplet was generated using the microfluidic device with two flow-focusing junctions. The prepared ATPS droplet has a diameter of 66 m, resulting in a total droplet volume of 150 pL. In the case of a Janus droplet, each half has a volume of 75 pL.

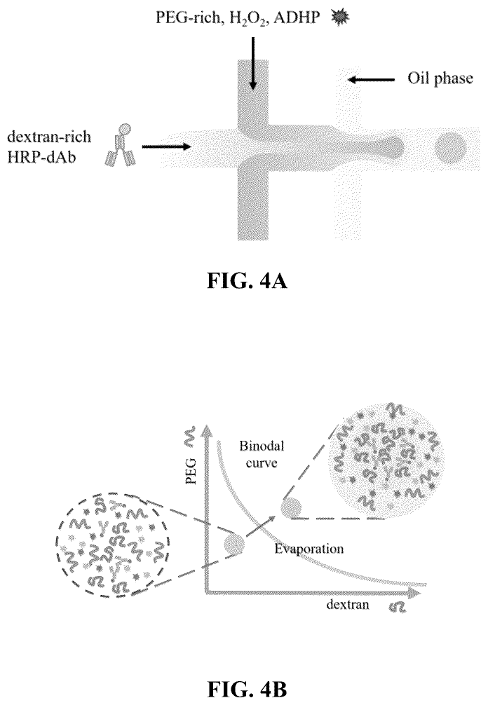

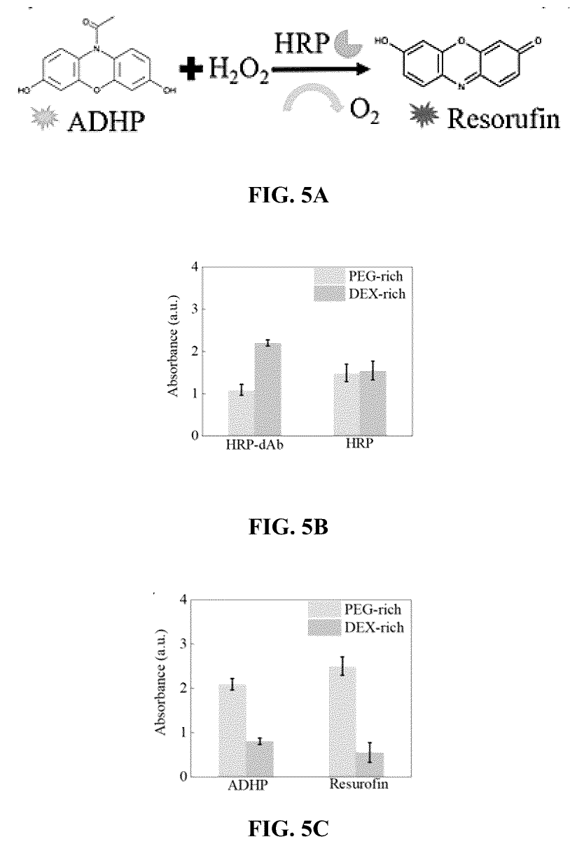

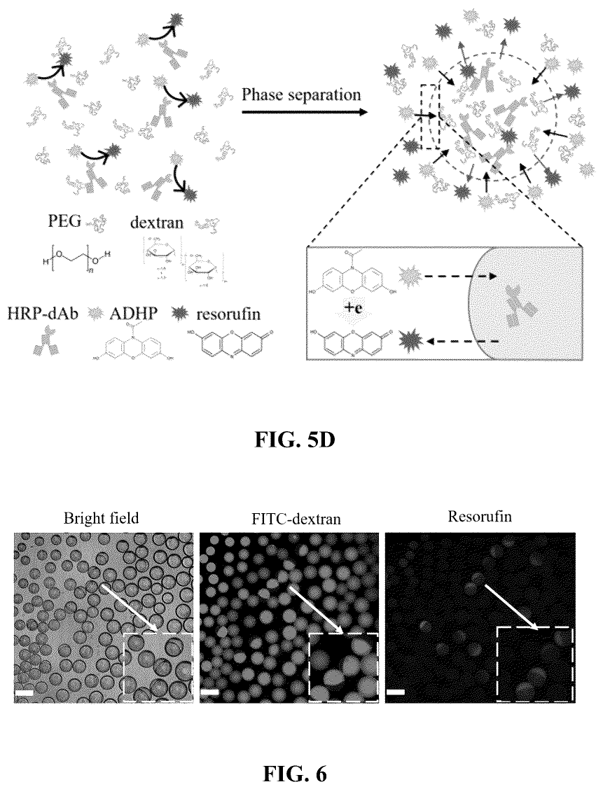

With the help of the microfluidic technique, the components of each enzyme-loaded ATPS droplet can be precisely tuned (FIG. 4A). Before droplet generation, the substrate (ADHP and H₂O₂) was dissolved in the PEG-rich phase at the volume ratio of 1:1, and the enzyme HRP conjugated antibody was dissolved in the dextran-rich phase at the volume ratio of 1:1. In the presence of HRP, the hydrogen peroxide (H₂O₂) could oxidize the non-fluorescent ADHP immediately into soluble, highly fluorescent resorufin (FIG. 5A). This reaction scheme could be used to detect HRP or H₂O₂ and be used in many immunoassays (ELISA), and cascade enzymatic reactions.

After that, the two aqueous phases met and mixed inside the microfluidic channel. Finally, the outermost phase of volatile hydrofluoroether (HFE) oil pinched the aqueous mixture into a single-phase droplet. After collecting the droplets, the phase separation of single-phase enzyme-loaded ATPS droplets was triggered by evaporation. FITC-dextran was used to label the dextran-rich phase and observe the non-equilibrium phenomena during the combined processes of liquid-liquid phase separation and reaction. In the control experiments, a series of tests were conducted by substituting the enzyme and substrate with pure buffer solutions, maintaining the molecular weights and mass fractions of PEG and dextran consistent with those utilized in the enzyme-loaded ATPS droplets. This approach enabled the assessment of the influence of enzymes and substrates on phase separation behavior and internal droplet dynamics while maintaining other experimental parameters constant.

### Example 5

### Characterization of Bulk ATPS

As illustrated in FIG. 4B, when the compositions of PEG and dextran existed below the binodal curve, the PEG-dextran-water mixture was a single-phase solution. In contrast, when PEG and dextran were mixed at sufficiently high concentrations, the homogeneous solution separated into two phases. Because dextran had a larger density than PEG, the upper part was the PEG-rich phase, and the lower part was the dextran-rich phase. When the HRP-ADHP catalysis was triggered in the PEG-dextran system, the affinity partitioning property of ATPS endowed the reaction process with more complexity. Specifically, biomolecules involved in the reaction process may have different partitioning preferences in ATPS, thus leading to different results. For instance, although the pure HRP did not show an apparent partitioning preference between PEG and dextran phases, the HRP conjugated secondary antibody (HRP-dAb) preferentially attached to the dextran-rich phase due to the hydrophilic structures in the antibody (FIG. 5B). In contrast, the substrate ADHP and catalysis product resorufin stayed in the PEG-rich phase due to their hydrophobicity (FIG. 5C). The partitioning preference of resorufin also be confirmed in Janus PEG-dextran droplets that the fluorescence generated by resorufin was localized in the PEG-rich phase (FIG. 6). Consequently, the enzymatic reaction happened at the PEG and dextran interfaces, where the ADHP and HRP-dAb met more frequently (FIG. 5D).

The partitioning affinities of biomolecules in ATPS systems can be quantified in bulk-phase and droplet-based systems. In the bulk phase experiments, the real-time formation of enzymatic products was monitored fluorescently (excitation: 550 nm, emission: 590 nm) in 96-well plates (the black type) by the SpectraMax iD3 multi-mode microplate reader (Molecular Devices, CA) at room temperature.

Since the absorption and fluorescence of resorufin were pH-dependent, the pH values of reacting solutions under various working conditions were characterized. MES buffer also called 2-(N-morpholino) ethanesulfonic acid, which is a monoprotic weak acid with a pKa of approximately 6.15, suitable for buffering within the pH range of 5.5 to 6.7. The results showed that the pH level for all the testing groups was stable and well-controlled under the 10 mM MES buffer (pH=6.0) (FIG. 3). Additionally, in the droplet-based methods, partitioning of resorufin could be directly observed through the fluorescence distribution in Janus ATPS droplets.

To calculate the concentration of catalysis products in aqueous solutions, the standard curves of resorufin were measured using the microplate reader. In detail, the stock solution of resorufin (5 mM) was prepared by dissolving them in DI water. The diluted solutions with known concentrations (0.1-1 mM) were dissolved using 10 mM MES buffer (pH=6.0), PEG, and dextran solutions separately (Table 5 and FIG. 3).

TABLE 5
Fitting curve parameters

| Buffer compositions | Equation | R² |
|---|---|---|
| 10 mM MES | y = 1.13 E8 × x + 6879046.32 | 0.995 |
| 12.5 mM PEG, 2 mM dextran | y = 1.22 E8 × x + 10000700 | 0.998 |
| in 10 mM MES | | |
| 12.5 mM PEG, 4 mM dextran | y = 1.28 E8 × x + 10002800 | 0.991 |
| in 10 mM MES | | |
| 12.5 mM PEG, 8 mM dextran | y = 1.41 E8 × x + 10001000 | 0.999 |
| in 10 mM MES | | |

The turnover number (k_{cat}) referred to the maximum number of chemical conversions of substrate molecules per second that a single active site would execute for a given enzyme concentration for enzymes with two or more active sites. For enzymes with a single active site, k_{cat} was treated as the catalytic constant. It could be calculated from the maximum reaction rate Vₘₐₓ and catalyst site concentration [E_{T}] as follows:

$${k}_{cat} = \frac{{V}_{max}}{[ {E}_{T} ]} \qquad ( 1 )$$

The k_{cat} could also be estimated based on the known enzyme concentration, the slope of the catalytic production curves versus time in linear regime at time point t as follows:

$${k}_{cat} = \frac{{[ Resorufin ]}_{t}}{t \cdot [ E ]} \qquad ( 2 )$$

To obtain the slope of the catalytic production curves versus time, the fluorescence intensities curves versus time needed to be transferred into the production concentration form. Thus, the concentration-fluorescent intensity calibration curves of enzymatic products must be measured in advance.

### Example 6

### Enrichment of N Protein in PEG/Dextran ATPS

Turning to FIGS. 4A-4B, which illustrated the basic principle of the target antigen being enriched into one of the two phases during the phase separation of ATPS. Intuitively, the hydrophilic bottom phase (e.g., dextran-rich phase) attracts the hydrophilic N protein more than the hydrophobic upper phase (e.g., PEG-rich phase). To quantitively investigate the antigen partitioning affinity under various conditions, fluorescent labels were used to indicate the partitioning preferences of antigen between two phases. The PEG-dextran ATPS was prepared as described in Example 3. Specifically, after labeling the N protein with fluorescein (FITC), the partitioning trend of N protein between the two phases was evaluated using the distribution ratio (DR). Evaluating DR provides qualitive analysis of N protein partitioning affinities. The calculation formula of DR is as follows:

$$Distribution ratio ( DR ) = \frac{{I}_{B}}{{I}_{T}}$$

I_{B}: Fluorescence intensity in bottom phase
I_{T}: Fluorescence intensity in top phase

### Example 7

Various parameters may influence the protein partitioning. In the following example, buffer pH values, ATPS types, polymer molecular weights, and polymer mass fractions were investigated in sequence.

### Effect of Various pH Value of Buffers on Antigen Enrichment

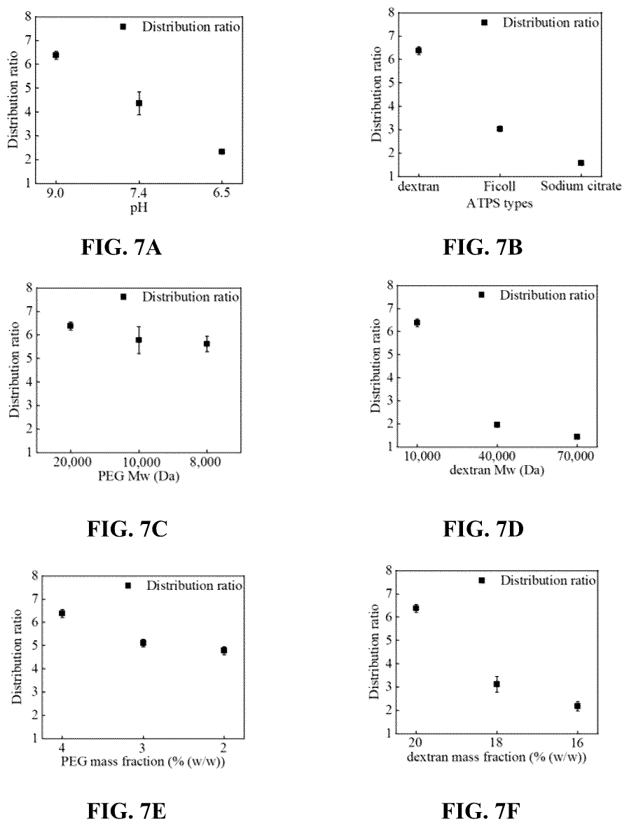

Firstly, ATPS constructed by PEG and dextran are dissolved by buffers with various pH values. Results showed that alkaline solution is more favorable for concentrating N protein into dextran-rich phase (FIG. 7A).

### Effect of Various ATPS Types on Antigen Enrichment

Three types of ATPS were provided as mentioned in Example 3. The upper phase was PEG-rich phase, and the bottom phase was dextran, Ficoll, or sodium citrate. PEG and dextran were two common crowding reagents that could influence molecular conformations and molecular organizations. The results showed that choosing dextran as the bottom phase leaded to higher antigen partitioning compared to Ficoll and sodium citrate (FIG. 7B).

### Effect of Various Molecule Weight of Two Phases on Antigen Enrichment

Besides, the to-be-partitioned protein was more concentrated by smaller molecules and more repelled by larger polymer molecules when all other factors (e.g., polymer concentration, salt composition, temperature, and other factors) were kept constant. It was observed that the antigen preferred more in the bottom phase when the PEG molecular weight was increased, and the dextran molecular weight was decreased. (FIGS. 7C-7D).

### Effect of Mass Fractions of Two Phases on Antigen Enrichment

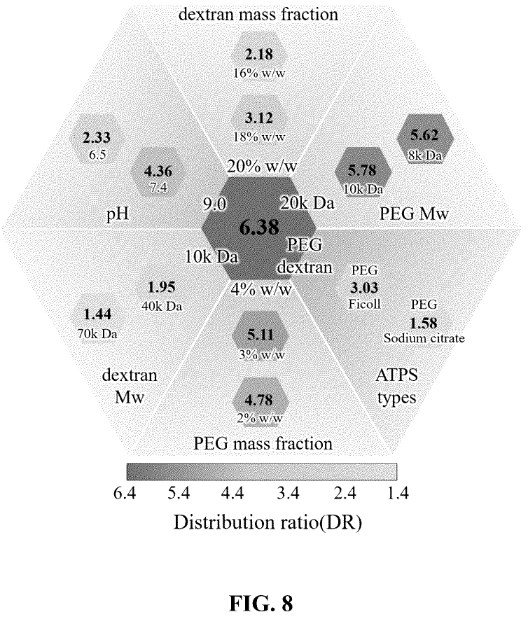

Likewise, increasing dextran mass fractions and decreasing PEG mass fractions resulted a higher partitioning preference towards the bottom phase (FIGS. 7E-7F). All the partitioning affinities could be combined in one figure to provide a multi-dimensional selection picture, as shown in FIG. 8.

The parameter evaluation process described above represented a universal methodology. Other conditions could also be involved when considering various antigens and ATPSs.

### Example 8

### Influence of PEG-Substrate Interactions on Reaction Kinetics

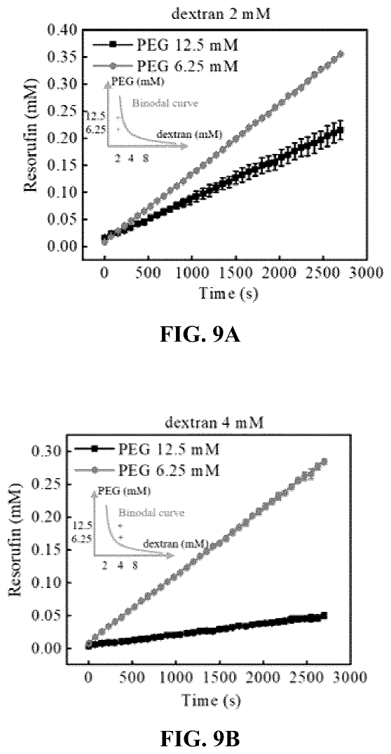

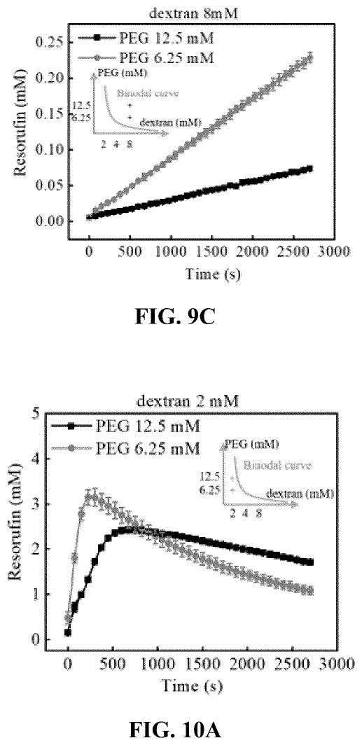

Initially, a constant substrate load was maintained while investigating the enzymatic reaction kinetics at relatively low enzyme concentrations (C_{HRP-dAb}=5.56 pM). Under these conditions, an excess substrate was available for enzymatic catalysis during the observation period of 45 minutes. The linear progression observed in the temporal trend of resorufin production provided further support for this. The results manifested a universal trend across all dextran concentrations. An augmentation in PEG concentration imposed an inhibitory effect on resorufin generation. This was borne out by the diminished slopes observed in the corresponding temporal trajectories when the PEG concentration was at the level of 12.5 mM (FIGS. 9A-9C and Table 6).

TABLE 6
the catalytic turnover rates (k_{cat}) under various conditions.

| Turnover | dextran | dextran | dextran |
|---|---|---|---|
| rate k_{cat} | 2 mM | 4 mM | 8 mM |
| PEG | 1428.62 ± | 326.09 ± | 486.28 ± |
| 12.5 mM | 7.16 | 4.99 | 2.20 |
| PEG | 2374.82 ± | 1899.60 ± | 1520.92 ± |
| 6.25 mM | 13.94 | 7.47 | 10.60 |

The data explicitly revealed a pronounced decrement in k_{cat} concomitant with an elevation in PEG concentration from 6.25 mM to 12.5 mM. Interestingly, this PEG-induced modulation of enzymatic activity retained its tenacity even as the dextran concentration oscillates between values from 2 mM to 8 mM.

PEG compounds are common reagents to induce molecular crowding conditions in vitro. Interactions between PEG and enzymes or substrates can decrease the enzymatic reaction significantly. Specifically, PEG polymer can interfere with hydrophobic interactions of protein and potentially destroy enzymes.

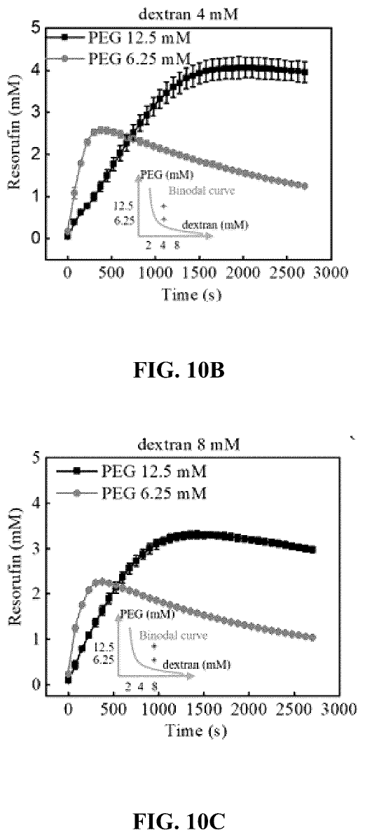

When the enzyme concentration is sufficiently high to exhaust the available substrate within the designated 45-minute observation period (C_{HRP-dAb}=555.56 pM), the temporal progression of resorufin production may eventually plateau, after attaining their maximal levels (FIGS. 10A-10C and Table 7).

The results once again demonstrated that an increase in PEG concentration consistently corresponded to a decrease in resorufin biosynthesis. This consistency in trends underscored the pervasiveness of PEG-substrate interactions across a range of enzymatic conditions, thereby reinforcing the complexity and significance of these biophysical interactions in the present system. Beyond this phenomenon, sustained activity after the reaction peak was observed. Specifically, when the PEG concentration was elevated to 12.5 mM, the catalytic activity maintained beyond the maximum reaction point. This was mirrored by the relative stability observed in the resorufin production trajectories, indicating an unanticipated endurance of enzymatic activity under these high PEG concentrations (FIGS. 10A-10C). When the PEG concentration was decreased to 6.25 mM, the generation of resorufin was diminished after reaching the maximum production point. This diminution in product generation was reflected by the downward trend observed in the corresponding temporal curves, highlighting the profound impact of PEG concentration on the sustained efficacy of enzymatic reactions.

TABLE 7
the catalytic turnover rates (k_{cat}) under various conditions.

| Turnover | dextran | dextran | dextran |
|---|---|---|---|
| rate k_{cat} | 2 mM | 4 mM | 8 mM |
| PEG | 6780.70 ± | 4440.87 ± | 4173.84 ± |
| 12.5 mM | 148.27 | 27.78 | 35.90 |
| PEG | 22822.97 ± | 11644.66 ± | 11342.23 ± |
| 6.25 mM | 1346.00 | 418.38 | 459.21 |

From Table 7, the turnover rates concurrently declined with an upsurge in PEG concentration. Furthermore, an analogous decrement in turnover rates was discernible as dextran concentrations ascend, underscoring the pervasive influence of crowder concentration on enzymatic kinetics.

It could be inferred that ATPS-forming materials, represented in the model by PEG and dextran, exerted a distinct influence on enzymatic kinetics. A general inhibitory impact of escalating PEG concentrations on resorufin generation was observed, existing across all studied dextran concentrations. Shifting the focus to scenarios involving higher enzyme concentrations, the temporal resorufin production trends encountered a phase of saturation, indicative of a dynamic shift in enzymatic activity. This unexpected observation underscored the intricate interplay between enzymatic activities and crowding agents in ATPS.

### Example 9

### Effect of Changing the Volume Ratios of Two-Phases on Antigen Enrichment

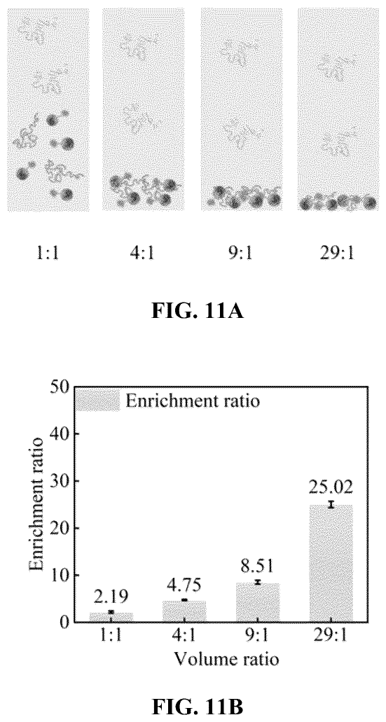

FIGS. 11A-11B depicts antigen enrichment by adjusting the two-phase volumetric ratios. To advance further the quantitative measurements of how many folds the target antigen would be enriched into the bottom phase, the enrichment ratio (ER) of partitioned proteins was calculated, which was defined as protein concentration in bottom phase (C₂) divided by the initial concentration before phase separation (C₁):

$$Enrichment Ratio ( ER ) = \frac{{C}_{2}}{{C}_{1}}$$

C₁: Antigen concentration in single phase
C₂: Enriched antigen concentration in bottom phase

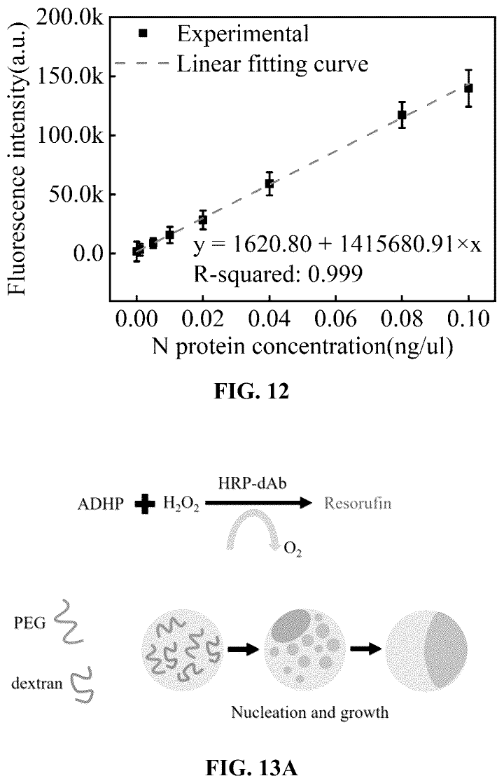

The initial concentration C₁ was recorded when diluting the fluorescent proteins, while the enriched concentration C₂ was calculated using the N protein standard calibration curve (FIG. 12).

Better enrichment performance could be achieved by enlarging the PEG-rich to dextran-rich ratios (FIG. 11B). For instance, when the volumetric ratios increase from 1:1 to 29:1, the ER scales up from 2.19 to 25.02. From the above results, it can be seen that adjusting the two-phase volume ratios can optimize the antigen enrichment fold.

### Example 10

### Microscale Flow Inside an Enzyme-Loaded ATPS Droplet

Four groups of experiments were conducted, each with varying compositions of enzyme-loaded ATPS droplets:
Case 1: Enzymatic reaction inside ATPS droplet;
Case 2: Without enzymatic reaction by removing the HRP-dAb;
Case 3: Without enzymatic reaction and the substrate;
Case 4: Control group using PEG and dextran in buffer.

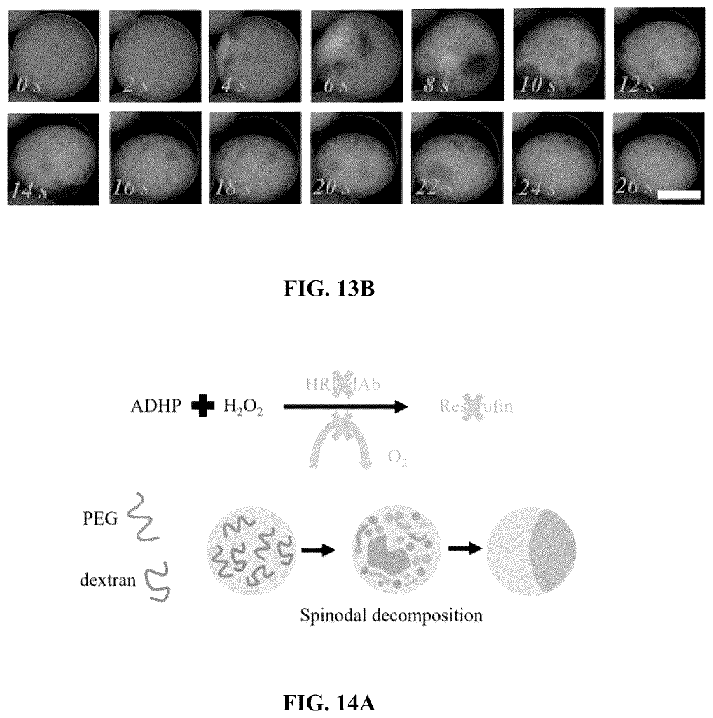

In case 1, the enzymatic reaction and phase separation occurred concurrently. The droplet underwent a classic nucleation and growth process, during which the dextran phase concentrated initially, followed by the emergence of PEG sub-droplets throughout the evaporation process (FIG. 13A). The phase separation process was observed and recorded using a fluorescence microscope (FIG. 13B). Intriguingly, the time taken for the droplet to transition from a single-phase to a two-phase morphology was shorter than that of the control group in case four, suggesting that the enzymatic reaction had accelerated the phase separation. Additionally, the internal flow within the droplet was enhanced, likely due to the oxygen bubbles generated by the catalytic reaction.

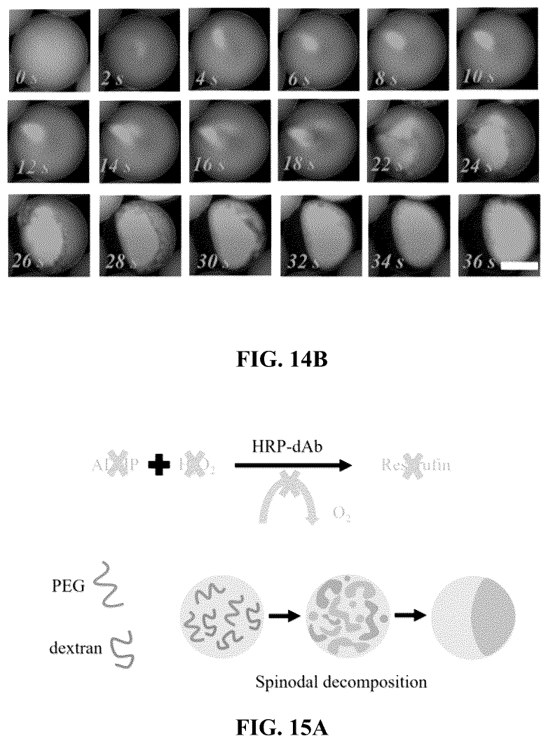

In case 2, the HRP-dAb was not involved, and no enzymatic reaction happened. In comparison to case one, the dextran-rich phase demonstrated a more notable concentration, evident by the emergence of a distinct lighter region within the initial ten seconds. Following this, the droplet experienced spinodal decomposition in the vicinity of the concentrated dextran-rich area. Another difference was the dextran sub-droplets emerge first (FIG. 14A). The phase separation process was observed and recorded using a fluorescence microscope (FIG. 14B). One possible explanation for this was that the H₂O₂ depolymerized the long dextran chains into shorter ones, thus transforming the dextran into the dispersed phase.

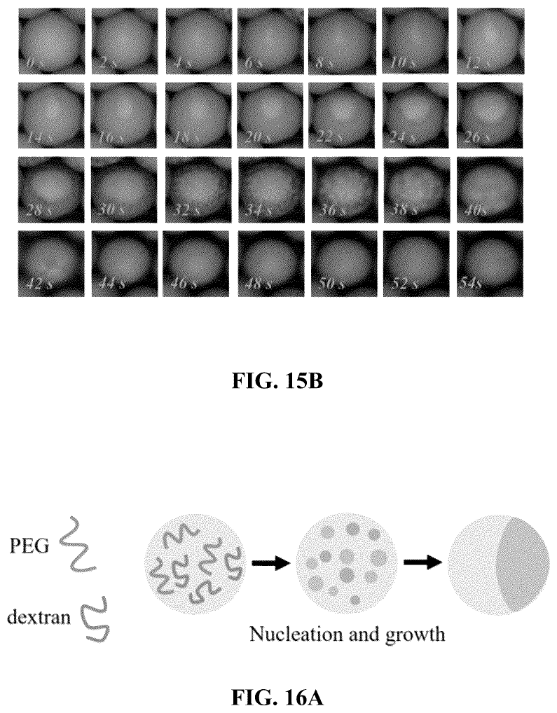

In case 3, the substrates (ADHP and H₂O₂) were not involved, and no enzymatic reaction taken place. As the dextran was concentrated, the dextran sub-droplets and PEG sub-droplets emerged simultaneously, and the mixtures underwent a characteristic spinodal decomposition process (FIGS. 15A-15B).

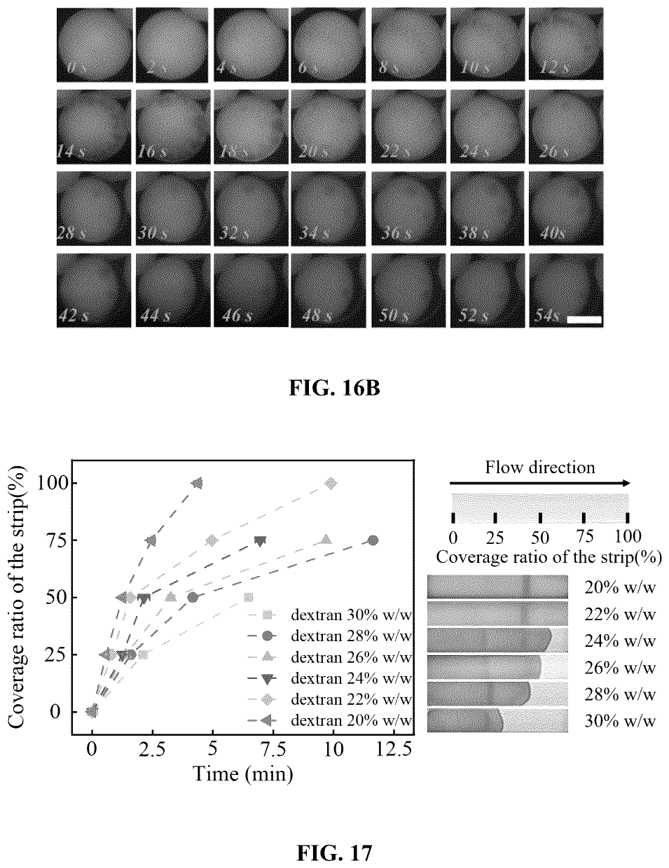

In case 4, the control group consisting of PEG and dextran in buffer solutions was prepared (FIG. 16A). The PEG sub-droplets emerged first, and an equivalent rise in concentration of the dextran phase comparable to cases one to three was not observed (FIG. 16B).

### Example 11

Flow Performance Optimization when Implementing ATPS-Enhanced RATs

The core components of rapid antigen test strips are nitrocellulose membrane, on which the sample solutions are loaded. According to the RATs user instructions, a smooth and complete flow of assay along the strip is the prerequisite of effective signal readouts. The flow performance within the porous membrane is driven by capillary force, which can be influenced by assay viscosity and other factors. When involving ATPS-forming materials into detecting assay, the addition of polymers or salts may change the solution viscosity and affect the flow property.

Therefore, the present invention compared the assay flow effectiveness by recording the total time needed for assay liquid to cover the whole test strips (FIG. 17). Dextran solutions with various concentrations were prepared for testing. It was natural to observe that increasing dextran concentrations may inhibit assay fluidity. When dextran mass fraction was lower than 22% w/w, assay liquids could cover the whole test strips within 15 minutes without collapsing the control lines. Besides, reducing the solution viscosity could shorten the cumulative time, which promoted the biomolecule incubation process. The relationship between polymer concentration and flow performance is an important factor to be considered when implementing the rapid antigen tests.

In one embodiment, the dextran mass fraction is in a range of about 0 wt % to about 20 wt %, and the PEG mass fraction is in a range of about 0 wt % to about 2 wt %.

### Example 12

### Comparison Between the Present Invention and Commercial Kits

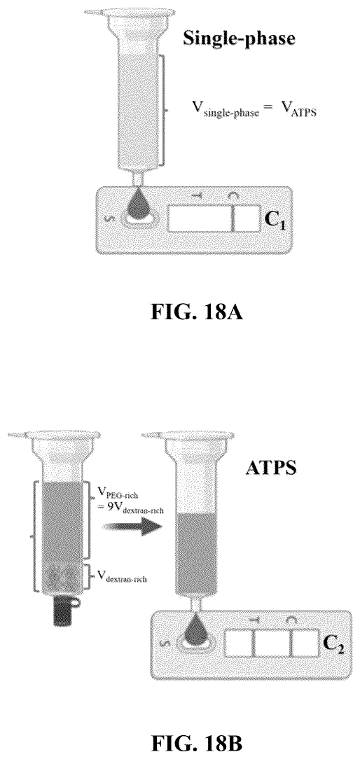

To validate the sensitivity enhancement via phase separation-based enrichment, the present invention also performed SARS-CoV-2 RAT assays with two commercial kits. For single-phase control experiments, the reconstituted SARS-CoV-2 antigen dilutions were prepared using various concentrations (0.064 pg/μl to 5000 pg/μl) and 10 mM HEPES buffer solution. For ATPS enrichment assays, the antigens were diluted in recombined ATPS mixtures with PEG-rich to dextran-rich volumetric ratio of nine, maintaining the same concentrations as single-phase groups (FIGS. 18A-18B).

The finalized phase separation system was successfully implemented in four commercialized RATs, three for detecting severe acute respiratory syndrome coronavirus 2 (SARS-CoV-2) and one for influenza virus.

### SARS-CoV-2:

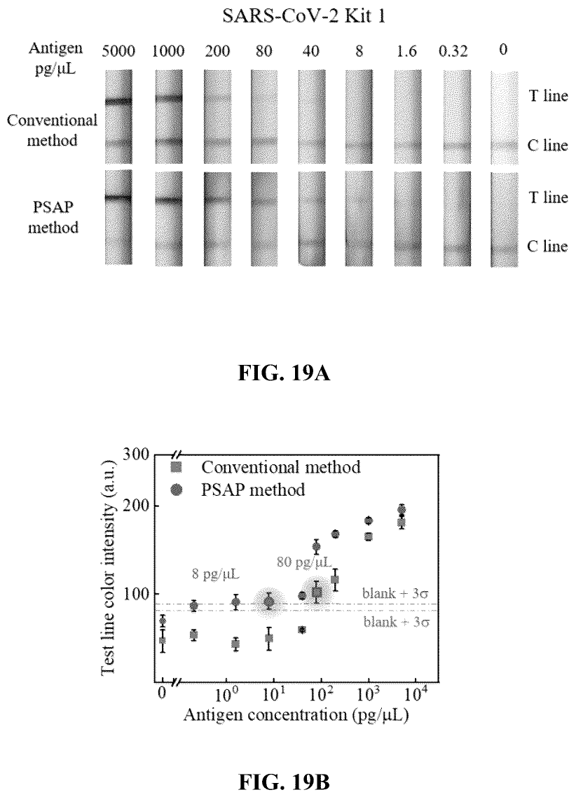

FIGS. 19A-19B depicted the detection sensitivity comparison between the present phase separation-assisted preconcentration (PSAP) technology and a commercial method. The RATs images of Genrui brand were displayed in FIG. 19A. Naked-eye readouts showed that commercial assays cannot generate signals when antigen concentrations were lower than 60 pg/μl. In contrast, after enhancement by phase separation, the limit of detection (LOD) of the present invention could achieve 8 pg/μl, resulting in a 7.5-fold sensitivity enhancement. The quantitative results were plotted using test line gray values (FIG. 19B), and the signal intensity of blank plus three times standard error (blank+3δ) was treated as the threshold for determining LOD.

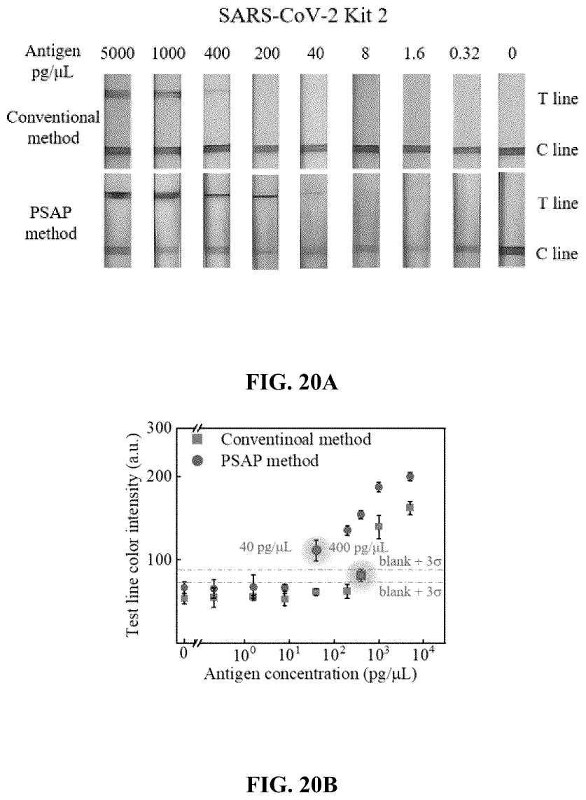

Next, another type of commercial RAT, the Banitore kit, was tested using the same experimental setups. FIGS. 20A-20B depicted the detection sensitivity comparison between the present invention and a commercially available brand (Banitore brand). The LOD of single-phase readouts was 200 pg/μl, while the ATPS-enhanced RATs of the present invention reached around 20 pg/μl (FIG. 20A). The 10-fold sensitivity enhancement was validated in FIG. 20B.

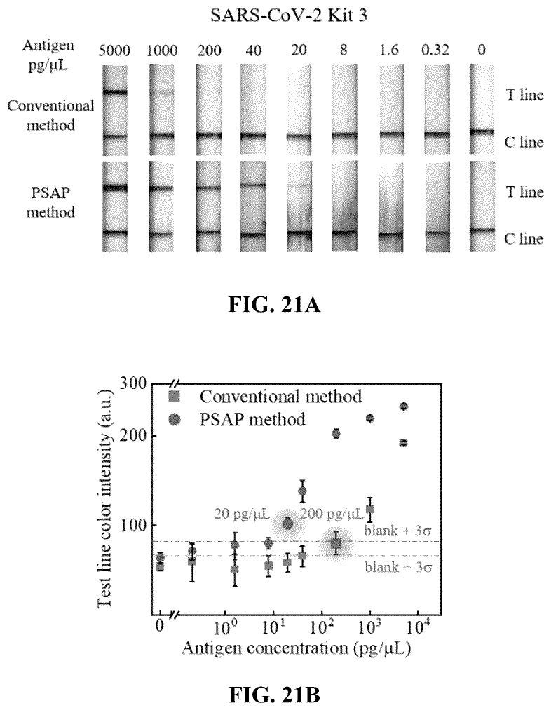

Furthermore, a third commercial RAT was tested using the same experimental setups. FIGS. 21A-21B depict the detection sensitivity comparison between the present phase separation-assisted preconcentration (PSAP) technology and a commercial method. The LOD of single-phase readouts was 200 pg/μl, while the ATPS-enhanced RATs of the present invention reached around 20 pg/μl (FIG. 21A). The 10-fold sensitivity enhancement was validated in FIG. 21B.

### Influenza A/B Virus:

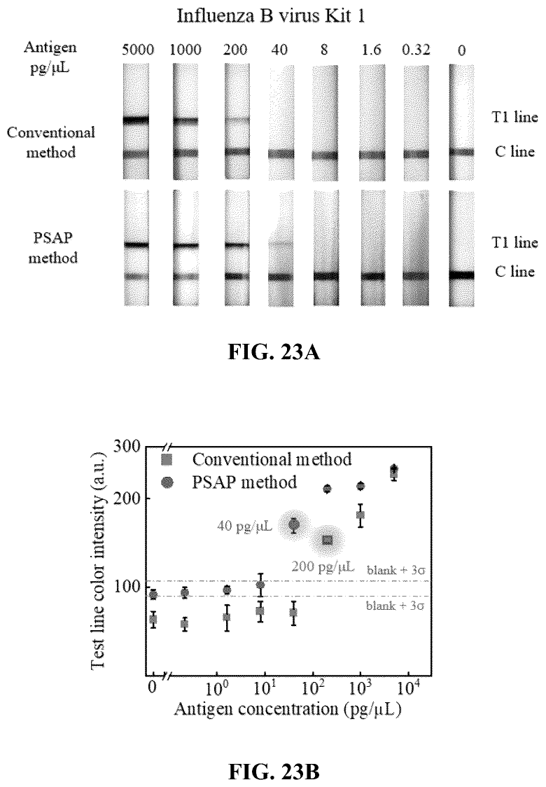

A first commercial RAT was tested using the same experimental setups, with the only difference being that the target for detection was changed to influenza B viruses. FIGS. 23A-23B depict the detection sensitivity comparison between the present phase separation-assisted preconcentration (PSAP) technology and a commercial method. The LOD of single-phase readouts was 40 pg/μl, and the ATPS-enhanced RATs of the present invention also reached around 40 pg/μl.

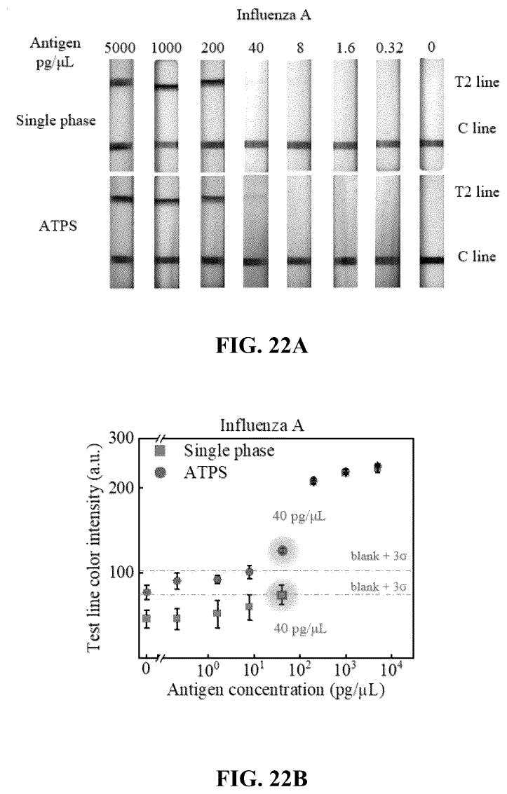

A second commercial RAT was tested using the same experimental setups, with the only difference being that the target for detection was changed to influenza A viruses. FIGS. 22A-22B depict the detection sensitivity comparison between the present phase separation-assisted preconcentration (PSAP) technology and a commercial method. The LOD of single-phase readouts was 200 pg/μl, while the ATPS-enhanced RATs of the present invention reached around 40 pg/μl (FIG. 22A). The 5-fold sensitivity enhancement was validated in FIG. 22B.

In summary, the LOD of viral nucleocapsid proteins was improved by up to 10-fold for SARS-CoV-2 and 5-fold for influenza B viruses, highlighting the generality of the PSAP technology. With universality and tunability, the PSAP technology was expected to be employed for diagnosing various diseases, extending beyond COVID-19 and seasonal influenza, ultimately broadening the scope and impact of RATs in the point-of-care fields.

### Example 13

Validation of Deactivated SARS-CoV-2 Viruses Cultured from Clinical Specimens

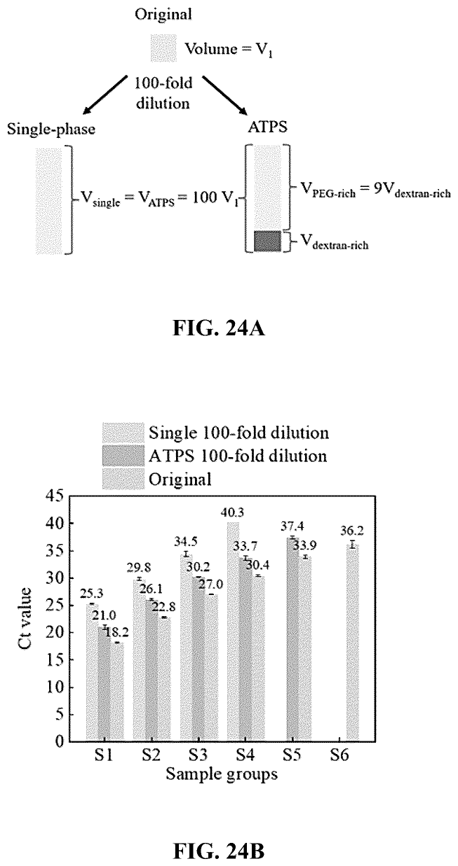

The present invention further investigated the clinical translational potential of the proposed method. Six groups of deactivated SARS-CoV-2 viruses cultured from clinical specimens of COVID-19 PCR-positive patients were tested (S1 to S6 groups). The Ct values of these cultured viruses were 18.2, 22.8, 27.0, 30.4, 33.9, and 36.2, respectively. Viral samples were aliquoted and stored in −80° C. refrigerator. Before testing the viral samples using commercial rapid antigen test kits, the working assays were prepared by constructing 100-fold dilutions using single-phase buffers and dextran-rich solutions separately. A 100-fold dilution step was involved during assay preparation process (FIG. 24A), leading to increased Ct values both in single-phase and ATPS groups (FIG. 24B and Table 8). For 100-fold dilution of ATPS, the antigens were diluted in recombined ATPS mixtures with PEG-rich to dextran-rich volumetric ratio of nine, maintaining the same concentrations as single-phase groups.

TABLE 8
Summary of the Ct values and RATs readouts of
deactivated viruses in single phase and ATPS

| | | Ct value | | Ct value | |
|---|---|---|---|---|---|
| | | 100-fold | | 100-fold | |
| Sample | Ct value | dilution | Single- | dilution | |
| groups | Original | Single-phase | phase RAT | ATPS | ATPS-RAT |
| S1 | 18.2 | 25.3 | Positive | 21.0 | Positive |
| S2 | 22.8 | 29.8 | Negative | 26.1 | Positive |
| S3 | 27.0 | 34.5 | Negative | 30.2 | Positive |
| S4 | 30.4 | 40.3 | Negative | 33.7 | Positive |
| S5 | 33.9 | ND | Negative | 37.4 | Positive |
| S6 | 36.2 | ND | Negative | ND | Positive |

*ND: not detected or detected with Ct > 40

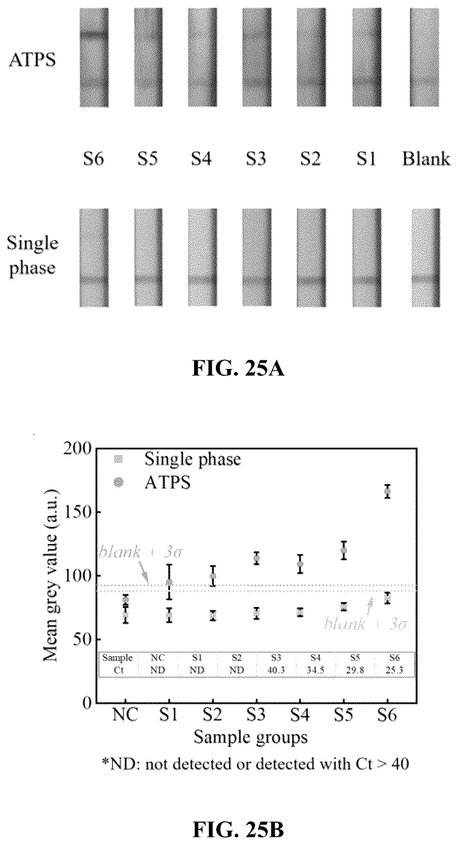

After that, RATs were performed using single-phase and ATPS-enriched solutions. The RATs for deactivated SARS-CoV-2 cultured virus were performed by pipetting a 100-μL aliquoted working assays to the sample loading area of the test strips. Both the single-phase and the ATPS-enriched sample solutions were prepared and tested within one day. The highest Ct value that can be detected in single-phase is 25.3, while the ATPS-enhanced RATs can distinguish sample with Ct equals 40.3 (FIGS. 25A-25B).

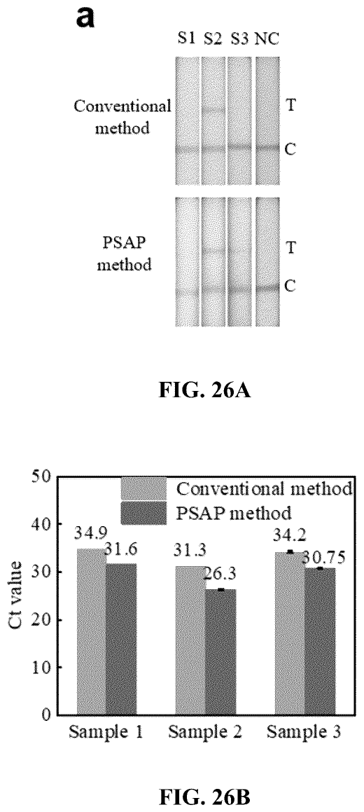

In another example, three groups of clinical samples collected from clinical specimens of COVID-19 PCR-positive patients were tested (S7 to S9 groups). The Ct values of these cultured viruses were 25.85, 19.55 and 24.25, respectively. Viral samples were aliquoted and stored in −80° C. refrigerator. Before testing the viral samples using commercial rapid antigen test kits, the working assays were prepared by constructing 50-fold dilutions using single-phase buffers and dextran-rich solutions separately (Table 9). Referring to FIGS. 26A-26B, the highest Ct value that could be detected using the conventional method was 31.3, while the ATPS-enhanced RATs could distinguish samples with Ct equal to 34.2.

TABLE 9
Summary of the Ct values and RATs readout of
clinical samples in single phase and ATPS

| | | Ct value in | | Ct value in | |
|---|---|---|---|---|---|
| | | single-phase | Single- | ATPS | |
| Sample | Ct value | (500-fold | phase | (50-fold | ATPS- |
| groups | Original | dilution) | RAT | dilution) | RAT |
| S7 | 25.85 | 34.9 | Negative | 31.6 | Negative |
| S8 | 19.55 | 31.3 | Positive | 26.3 | Positive |
| S9 | 24.25 | 34.2 | Negative | 30.75 | Positive |

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### INDUSTRIAL APPLICABILITY

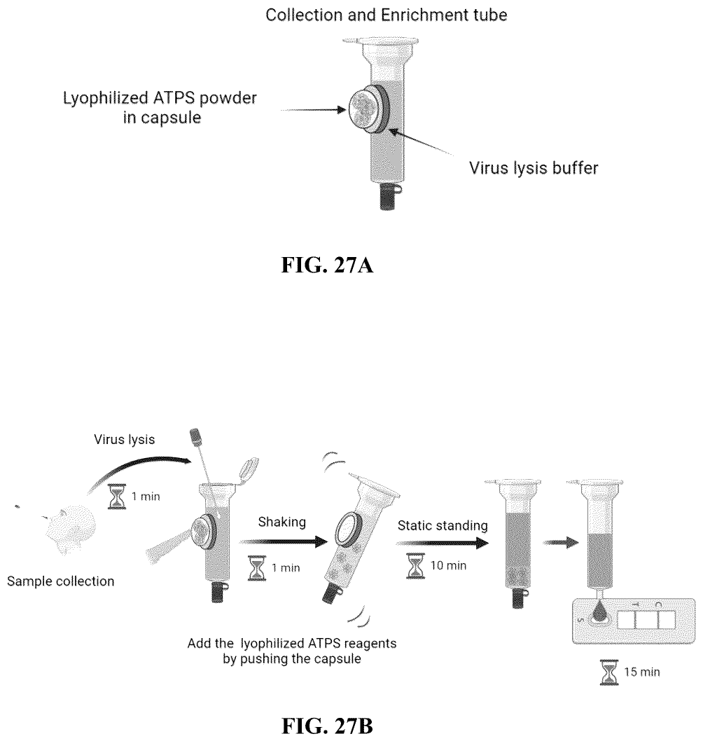

The present invention provides a novel method for detecting N protein from SARS-CoV-2 virus in a sample. Besides, the present invention proposes a novel design to integrate and automate the sample collection and analyte enrichment process. The device can be directly used in the commercial lateral flow assay-based rapid antigen test kits, without additional modification to the existing test strips. The design of the tube, and the testing process are shown in FIGS. 27A-27B.

### Definition

The term “Janus droplet” is a special type of microscale droplet that possesses two different chemical or physical properties. Typically, Janus droplets are composed of two immiscible liquids, each occupying a distinct region within the droplet. These droplets often exhibit asymmetry, with one side being hydrophilic and the other hydrophobic. Various methods can be employed to prepare Janus droplets, including the use of specialized surfactants, microfluidic techniques, or specific preparation conditions.

The “enrichment ratio” refers to the increase in concentration of a target substance or element relative to the original sample or background concentration during a particular treatment process. This term is commonly used to describe the degree of concentration increase during extraction, separation, or detection processes. The enrichment ratio is typically expressed as a numerical multiplier or percentage to quantify the extent of concentration of the target substance during the treatment process.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of ±10%, ±5%, ±1%, or ±0.5% of the given value or range. The range may be indicated herein as from one endpoint to another endpoint or between two endpoints. Unless otherwise specified, all the ranges disclosed in the present disclosure include endpoints. The term “substantially coplanar” may refer to two surfaces within a few micrometers (μm) positioned along the same plane, for example, within 10 μm, within 5 μm, within 1 μm, or within 0.5 μm located along the same plane. When reference is made to “substantially” the same numerical value or characteristic, the term may refer to a value within ±10%, 5%, ±1%, or ±0.5% of the average of the values.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### REFERENCES

The disclosures of the following references are incorporated by Reference
1. R. Y. Chiu, A. V. Thach, C. M. Wu, B. M. Wu, D. T. Kamei, An Aqueous Two-Phase System for the Concentration and Extraction of Proteins from the Interface for Detection Using the Lateral-Flow Immunoassay. PLoS One 10, e0142654 (2015).
2. E. Jue, C. D. Yamanishi, R. Y. Chiu, B. M. Wu, D. T. Kamei, Using an aqueous two-phase polymer-salt system to rapidly concentrate viruses for improving the detection limit of the lateral-flow immunoassay. Biotechnol Bioeng 111, 2499-2507 (2014).
3. D. W. Bradbury et al., Automation of Biomarker Preconcentration, Capture, and Nanozyme Signal Enhancement on Paper-Based Devices. Anal Chem 91, 12046-12054 (2019).

What is claimed is:

1. An ultrasensitive rapid antigen detection kit for detecting N protein from RNA viruses in a sample, comprising:
a sample collector for collecting the sample from a patient;
a collection and enrichment tube comprising
a first part containing a lyophilized reagent, and
a second part containing a lysis buffer; and
a rapid antigen detection component comprising a test strip; and
a manual,
wherein the sample is mixed with the lyophilized reagent and the lysis buffer to form at least one Janus droplet having enriched N protein,
wherein the test strip comprises a sample application port for applying the at least one Janus droplet from the collection and enrichment tube to the test strip, an absorbent pad for wicking the sample, a detection line for capturing the N protein, and a control line for indicating the validity of the detection.

2. The ultrasensitive rapid antigen detection kit of claim 1, wherein the lyophilized reagent comprises at least two polymers or a polymer containing a salt.

3. The ultrasensitive rapid antigen detection kit of claim 2, wherein a first polymer of the at least two polymers is polyethylene glycol with a molecule weight ranging from 1000 Da to 40,000 Da; and a second polymer of the at least two polymers is dextran with a molecule weight ranging from 8,000 Da to 100,000 Da, Ficoll with a molecule weight ranging from 50,000 Da to 400,000 Da, or a salt sodium citrate with a molecule weight of ranging from 100 Da to 400 Da, or a combination thereof.

4. The ultrasensitive rapid antigen detection kit of claim 3, wherein the first polymer and the second polymer have a volume ratio in a range of 1:1 to 29:1.

5. The ultrasensitive rapid antigen detection kit of claim 4, wherein the kit enhances an enrichment ratio of the N protein with a range of 2 to 5.

6. The ultrasensitive rapid antigen detection kit of claim 3, wherein the first polymer forms a hydrophobic upper phase, the second polymer forms a hydrophilic bottom phase, and the N protein is attracted by the hydrophilic bottom phase, and wherein the hydrophobic upper phase and the hydrophilic bottom phase are combined by a droplet microfluidic device with two flow-focusing junctions to generate the at least one Janus droplet.

7. The ultrasensitive rapid antigen detection kit of claim 6, wherein the at least one Janus droplet has a diameter of 50-70 μm, resulting in a total droplet volume of 150 pL.

8. The ultrasensitive rapid antigen detection kit of claim 6, wherein the hydrophobic upper phase further comprises 2 mM to 10 mM of 10-Acetyl-3,7-dihydroxyphenoxazine and H₂O₂, and the hydrophilic bottom phase further comprises 5 pM to 5000 pM of HRP-conjugated antibody.

9. The ultrasensitive rapid antigen detection kit of claim 1, wherein the lysis buffer comprises Triton X, sodium dodecyl sulfate (SDS) lysis buffer with dithiothreitol (DTT).

10. The ultrasensitive rapid antigen detection kit of claim 1, wherein the RNA viruses comprise coronaviruses, influenza viruses, Monkeypox viruses, human immunodeficiency viruses, respiratory syncytial viruses and lymphocytic choriomeningitis viruses.

11. The ultrasensitive rapid antigen detection kit of claim 1, wherein the kit is capable to discern samples with a Ct value of approximately 40.3.

12. A method for detecting N protein from RNA viruses in a sample, comprising the following steps:
collecting a sample from a subject suspected of being infected with the RNA viruses;
mixing the sample with a lyophilized reagent and a lysis buffer with a ratio within the range of 1:1 to 1:5 in a collection and enrichment tube to form at least one Janus droplet having enriched N protein; and
applying the at least one Janus droplet from the collection and enrichment tube to a test strip of a rapid antigen detection component to obtain the test result.

13. The method of claim 12, wherein the lyophilized reagent comprises at least two polymers or a polymer containing a salt.

14. The method of claim 13, wherein a first polymer of the at least two polymers is polyethylene glycol with a molecule weight ranging from 1000 Da to 40,000 Da; and a second polymer of the at least two polymers is dextran with a molecule weight ranging from 8,000 Da to 100,000 Da, Ficoll with a molecule weight ranging from 50,000 Da to 400,000 Da, or a salt sodium citrate with a molecule weight of ranging from 100 Da to 400 Da, or a combination thereof.

15. The method of claim 14, wherein the first polymer and the second polymer have a volume ratio in a range of 1:1 to 29:1.

16. The method of claim 15, wherein the kit enhances an enrichment ratio of the N protein with a range of 2 to 5.

17. The method of claim 14, wherein the first polymer forms a hydrophobic upper phase, the second polymer forms a hydrophilic bottom phase, and the N protein is attracted by the hydrophilic bottom phase, and wherein the hydrophobic upper phase and the hydrophilic bottom phase are combined by a droplet microfluidic device with two flow-focusing junctions to generate single-phase droplet.

18. The method of claim 12, wherein the lysis buffer comprises Triton X, sodium dodecyl sulfate (SDS) lysis buffer with dithiothreitol (DTT).

19. The method of claim 12, wherein the RNA viruses comprise coronaviruses, influenza viruses, Monkeypox viruses, human immunodeficiency viruses, respiratory syncytial viruses and lymphocytic choriomeningitis viruses.
